(19) United States
(12) Patent Application Publication
(10) Pub. No.: US 2012/0263812 A1
(43) Pub. Date: Oct. 18, 2012

(54) USE OF AN EXTRACT OF PUNICA GRANATUM FOR COMBATING CANITIES

(75) Inventor: Stephane Commo, Chaville (FR)

(73) Assignees: NESTEC SA, Vevey (CH); L'OREAL, Paris (FR)

(21) Appl. No.: 13/505,177

(22) PCT Filed: Oct. 29, 2010
(86) PCT No.: PCT/FR10/52327

§ 371 Date: Jun. 26, 2012

Related U.S. Application Data
(60) Provisional application No. 61/272,829, filed on Nov. 9, 2009.

(30) Foreign Application Priority Data
Oct. 30, 2009 (FR) 09 57663

(51) Int. Cl. A61K 36/185; A61P 43/00; A61K 36/52; A61K 36/73; A61K 36/87

(52) U.S. Cl. 424/765; 424/769; 424/766; 424/771

(57) ABSTRACT

The invention relates to the use of at least one plant of the species Punica granatum or an extract thereof as an agent for reducing or preventing the whitening of head hair and/or bodily hair, said extract being used orally.

The invention also relates to a cosmetic treatment process by administration of Punica granatum.

The present invention relates to cosmetic treatment processes for preventing or reducing whitening of the integuments, and to the use of plant extracts as agents for promoting the pigmentation of the integuments and/or for limiting and/or preventing depigmentation. The invention in particular concerns extracts of pomegranate, or compositions containing them, for combating canities.

The color of human hair and skin depends on various factors and especially the seasons of the year, race, sex and age. It is mainly determined by the concentration of melanin produced by the melanocytes. Melanocytes are specialized cells which, by means of particular organelles, the melanosomes, synthesize melanin.

Melanin synthesis or melanogenesis is complex and schematically involves the following main steps:

### Tyrosine--->Dopa--->Dopaquinone--->Dopachrome--->Melanin

Tyrosinase (monophenol dihydroxyl phenylalanine:oxygen oxidoreductase EC 1.14.18.1) is involved in this reaction sequence by especially catalysing the reaction for the conversion of tyrosine into dopa (dihydroxyphenylalanine) and the reaction for conversion of dopa into dopaquinone.

The upper part of the hair follicle is in the form of a tubular invagination of the epidermis which tunnels down to the deep layers of the dermis. The lower part, or hair bulb, itself comprises an invagination in which is located the dermal papilla. Around the dermal papilla, in the lower part of the bulb, is a region populated with cells with a high degree of proliferation (matrix cells). These cells are the precursors of the keratinized cells that will constitute the hair. The cells that result from the proliferation of these precursors migrate vertically in the bulb and become gradually keratinized in the upper part of the bulb, and this assembly of keratinized cells forms the hair stalk.

The color of head hair and bodily hair is based partly on the presence in variable amounts and ratios of two groups of melanins: eumelanins (brown and black pigments) and pheomelanins (red and yellow pigments). The pigmentation of head hair and bodily hair requires the presence of melanocytes in the bulb of the hair follicle. These melanocytes are active, i.e. they synthesize melanins. This melanin is transmitted to the keratinocytes intended to form the hair stalk that will result in the growth of a pigmented strand of head hair or bodily hair. This structure is known as a “follicular pigmentation unit”. In mammals, melanogenesis involves at least three enzymes: tyrosinase, DOPAchrome tautomerase (TRP-2) and DHICA oxidase (TRP-1). Tyrosinase is the enzyme that initiates the biosynthesis of melanins. It is also described as being the enzyme that limits melanogenesis. Tyrosinase catalyses the oxidation of tyrosine to dopa and then to dopaquinone. The compound dopaquinone becomes spontaneously transformed into dopachrome, or into cysteinyldopa derivatives in the presence of cysteine. TRP-2 catalyses the tautomerization of dopachrome to 5,6-dihydroxyindole-2-carboxylic acid (DHICA). In the absence of TRP-2, dopachrome undergoes a spontaneous decarboxylation to form 5,6-dihydroxyindole (DHI). Finally, TRP-1 oxidizes the DHICA compounds to form quinone derivatives. The three enzymes tyrosinase, TRP-2 and TRP-1 appear specifically involved in eumelanogenesis. Furthermore, the activity of these three enzymes was described as necessary for the maximum activity of eumelanin biosynthesis.

Head hair and bodily hair undergo a cycle. This cycle comprises a growth phase (anagenic phase), a degeneration phase (catagenic phase) and a resting phase (telogenic phase) after which a new anagenic phase develops. As a result of this hair cycle, the follicular pigmentation unit must also be cyclically renewed. In man, during the telogenic-anagenic transition, some of the inactive melanocytes contained in the telogenic capsule proliferate, position themselves around the dermal papilla of the nascent bulb and begin to express the enzymes necessary for the synthesis of melanins, such as tyrosinase and TRP-1, but not the enzyme TRP-2 (Pigment Cell Res, 2004 October; 17:488-497). In parallel, the rest of the quiescent melanocytes remain inactive, in the upper region of the hair follicle. The enzymes tyrosinase and TRP-1 are expressed in the melanocytes of the hair bulb throughout the anagenic phase, but are no longer expressed in the melanocytes during the catagenic phase and the telogenic phase. Thus, the normal cycle of the melanocytes in the human hair follicle requires the presence of precursor melanocytes in the upper region of the hair follicle, which will be cyclically activated to regenerate the follicular pigmentation unit.

It is known that in the majority of populations, the brown coloration of the skin and the maintenance of a constant coloration of the hair are important aspirations.

It is accepted that the appearance of gray or white bodily hair and/or head hair, or canities, is associated with a decrease of melanin in the hair stalk. This phenomenon arises naturally in the life of an individual. However, human beings seek to have a younger appearance and, for esthetic reasons, it is often attempted to combat this phenomenon, especially when it occurs at a relatively early age.

Numerous solutions have thus been proposed in the field of artificial coloration by supplying exogenous colorants aimed at giving the hair a color that is as close as possible to its natural color. Another approach consists in stimulating the natural pigmentation route.

Among the proposed solutions, mention may be made of compositions containing a phosphodiesterase inhibitor (WO 95/17161), DNA fragments (WO 95/01773), diacyl glycerol (WO 94/04122), prostaglandins (WO 95/11003) or pyrimidine 3-oxide derivatives (EP 829 260). Patent application WO 04/073 594 proposes the use of inhibitors of the enzyme 15-PGDH. EP 1 870 081 describes the use of ellagic acid or derivatives thereof for treating canities.

However, there is still a need for novel effective solutions for promoting the pigmentation of head hair and/or bodily hair and thus preventing or reducing canities.

Unexpectedly, it has now been found that this aim and others can be achieved by using the plant Punica granatum, fractions thereof or extracts thereof.

Consequently, one subject of the present invention is the use of at least one plant of the species Punica granatum and/or an extract thereof or a composition containing at least one such plant or an extract thereof, as an agent for reducing or preventing the whitening of head hair and/or bodily hair, said extract being used orally.

The pomegranate tree (Punica granatum) is a small fruit tree with deciduous foliage, originating from Asia, in particular from the Middle East, belonging to the Punicacea family. It may reach a height of about 5 m, but is often not more than 1.50 m. The flowers are orange-red or scarlet, and single or double, depending on the variety. The fruit is edible and has been consumed since Antiquity. Synonyms are Punica spinosa, Punica florida or Granatum puniceum.

Its fruit is the pomegranate, which is orange-red colored, with a hard, tough pericarp (or peel) containing 6 to 12 membranous lobes bearing numerous triangular seeds with a translucent, juicy aril. Pomegranate is rich in vitamins (B, C and D) and in polyphenols in the form of tannins. Peel extracts have been proposed for their astringent, antibacterial and anti-haemorrhagic properties. Tannin-rich extracts are traditionally used in the Moroccan pharmacopea for blackening the hair, as a topical application.

Patent EP 138 419 describes the topical application of pomegranate extract for prolonging the intensity of the color of dyed hair.

WO 2007/004 229 describes the use of oily fractions of pomegranate seeds for promoting skin regeneration.

FR 2 730 408 relates to compositions for depigmenting the skin by topical application, which may comprise extracts of various fruit, including that of Punica granatum.

EP 1 523 895 concerns oral compositions containing a combination of an extract of Punica granatum, a fatty acid, in particular conjugated linoleic acid (CLA), and OPC.

U.S. Pat. No. 6,630,163 describes compositions for treating dermatological disorders containing fruit extracts, and especially pomegranate extracts.

US 2006/0 280 819 describes food supplements prepared from pomegranate seeds. However, to the Applicants' knowledge, it has never been proposed to use Punica granatum or extracts thereof orally for preventing, limiting or stopping the progress of canities, and/or for promoting the natural repigmentation of the integuments, in particular of head hair and/or bodily hair.

One subject of the invention is thus especially the use of at least one plant of the species Punica granatum or an extract thereof or a composition containing at least one such plant or an extract thereof in a physiologically acceptable medium, as an agent for promoting and/or inducing and/or stimulating pigmentation of the integuments, and/or as an agent for preventing and/or limiting the depigmentation and/or whitening of the integuments, especially as an agent for preventing and/or limiting canities; this agent is more particularly used on mammals, in particular on man.

According to another of its aspects, the invention relates to the cosmetic use of at least one plant of the species Punica granatum or an extract thereof in a composition suitable for the oral route, for promoting and/or inducing and/or stimulating pigmentation of the integuments, and/or for preventing and/or limiting the depigmentation and/or whitening of the integuments and/or for preventing and/or limiting canities.

The invention relates to the cosmetic use of at least one plant of the species Punica granatum or at least one extract thereof for the preparation of a composition for promoting and/or inducing and/or stimulating pigmentation of the integuments, and/or for preventing and/or limiting the depigmentation and/or whitening of the integuments and/or for preventing and/or limiting canities.

The compositions that are useful according to the invention may contain a physiologically acceptable medium or excipients.

The term “skin” means all of the cutaneous coating, and especially the scalp and mucous membranes. The term “integuments” means all of the tegumental appendages and especially the nails, bodily hair and head hair. The terms “bodily hair” and “head hair” mean all of the pilous appendages and especially also the eyelashes and the eyebrows. The term “at least one” means one or two or more, and especially mixtures in all proportions of the various cited elements. Unless otherwise specified, the term “one” in the present patent application should be understood as meaning at least one.

The Punica granatum extract that is useful according to the invention may be chosen from the extracts of flowers, extracts of fruit, extracts of seeds and extracts of peel of Punica granatum, and mixtures thereof in all proportions. Advantageously, use is made of a fruit extract—or pomegranate extract—it being possible for this extract to originate from any part of the pomegranate or more specifically from the peel and/or the seeds. A whole extract may be used, i.e. an extract comprising all parts of the pomegranate, from which the ligneous parts have been removed. According to another embodiment of the invention, use will be made of at least one extract enriched in certain fractions and especially an extract with a polyphenol assay.

The pomegranate extract may be obtained from any part of the pomegranate tree and in particular from the fruit optionally including the seeds, or alternatively in particular from the fruit peel.

The term “extract” is understood to mean both a crude mixture of parts of the plant roughly broken into pieces and of the extraction solvent, and fractions or preparations, which are more or less processed, of active principles solubilized during the extraction. It is possible to use a total extract, that is to say an extract comprising all of the fractions present in the parts of Punica granatum, optionally freed of the cellulose parts. According to another embodiment of the invention, use will be made of at least one extract enriched in certain fractions.

The extract may be obtained by any method for preparing a plant extract known to those skilled in the art.

In particular, the extract may be obtained by macerating the part of the plant in water, or in a solvent composed of a mixture of water and an organic solvent, for example water-alcohol, or else water-acetone, or else water-propylene glycol, or else water-butylene glycol. The water-organic solvent ratio may vary. The extraction will be prepared, for example, in a 50% water-ethanol mixture or in a 20% water-80% ethanol solution. The plant/solvent ratio may vary, for example and with no limitation, from ¼ to 1/20. Advantageously, the preparation of the extract starts with the milling of the parts of the plant, followed by a maceration in the extraction solvent for several hours. The extraction may be carried out with stirring in order to improve the performance thereof. The extraction may be carried out at room temperature or by increasing the temperature, for example to 50° C. or else to 60° C. Once the extraction has been carried out, the solution is filtered.

The solution thus obtained may be concentrated by any process known to those skilled in the art. Likewise, the solution obtained may be lyophilized by any conventional lyophilization method; a powder is thus obtained.

The extract that is useful for the implementation of the invention may also correspond to pomegranate juice, obtained by pressing the fleshy parts of the fruit. This juice may undergo fermentation, concentration and/or freeze-drying steps and/or any operation for facilitating its conservation, such as sterilization treatments, known to those skilled in the art.

Extraction from the fruit of the pomegranate tree or shrub, including the seed, may lead, according to one particular embodiment, to the preparation of an essential oil. The extract in the form of a concentrated solution, and also the extract in powder form, and also the extract in the form of an essential oil may be taken up in a medium suitable for oral human consumption.

According to one advantageous embodiment of the invention, the extract is chosen from aqueous extracts and alcoholic or aqueous-alcoholic extracts of Punica granatum. Such extracts are recorded, for example, under the number CAS 84961-57-9 and sold, for example, by the company MMP Inc. under the trade name Pomegranate Juice Extract E40; other extracts are sold, for example, by the company Blue California under the trade name Pomegranate Extract 70% or by the company Naturex under the trade name Pomegranate Extract 40%, or alternatively by the company Guillin Layn Natural Ingredients under the trade name Pomegranate Seed P.E.

Advantageously, the extract of Punica granatum is present in a composition suitable for the oral route, containing physiologically acceptable excipients.

The compositions according to the invention are intended to be absorbed via any route that enables systemic passage, in particular the oral route, in order to protect or maintain the surface parts of the body in good condition, or to improve an individual's appearance, in particular that of his skin and its appendages. The compositions according to the invention are in particular food supplements.

Via the oral route, the composition according to the invention may contain the extract(s) of Punica granatum dissolved in a food liquid such as an optionally flavored aqueous or aqueous-alcoholic solution. They may also be incorporated in an ingestible solid excipient and be, for example, in the form of granules, pills, tablets or coated tablets. They may also be placed in solution in a food liquid that is itself optionally conditioned in ingestible capsules. For ingestion, numerous embodiments of oral compositions and especially of food supplements are possible. They are formulated via the usual processes for producing coated tablets, gel capsules, gels, emulsions, tablets, capsules or solutions. In particular, the active agent(s) according to the invention may be incorporated into any other form of food supplements or enriched foods, for example food bars, or compacted or non-compacted powders. The powders may be diluted with water, in soda, dairy products or soybean derivatives, or may be incorporated into food bars.

The active agents according to the invention may be formulated with the usual excipients and components for such oral compositions or food supplements, i.e. especially fatty and/or aqueous components, humectants, thickeners, preserving agents, texture agents, taste agents and/or coating agents, antioxidants, preserving agents and dyes that are common in the food sector.

The amount of Punica granatum plant or extract will be adapted by a person skilled in the art according to the nature of the active agent and the desired effect. In certain embodiments of the invention, the extract of Punica granatum may constitute up to 100% of the composition. As a guide, the extract of Punica granatum will be present in a composition suitable for the oral route in a concentration of between 0.01% and 99% by weight relative to the total weight of the composition, generally less than or equal to 90% and especially from 0.1% to 50% by weight. Concentrations of from 5% to 50% relative to the total weight of the composition and especially from 40% to 50% may thus be used in accordance with the invention.

The content of Punica granatum or of extract of Punica granatum in the compositions will be adapted so as to obtain a daily intake of between 10 mg and 1000 mg of extract of Punica granatum, especially between 200 mg and 500 mg.

The compositions or the extracts of a plant of the species Punica granatum according to the invention are used for reducing and/or preventing and/or retarding the natural whitening of keratin fibers that occurs physiologically in the course of aging, more particularly in man. The human keratin fibers to which the invention applies are especially head hair, the eyebrows, the eyelashes, beard hair, moustache hair and pubic hair. More especially, the invention applies to human head hair and/or eyelashes.

A subject of the invention is also a plant of the species Punica granatum or an extract thereof for its use for preventing and/or treating canities.

In particular, the invention is directed toward limiting, preventing or stopping the disappearance of the melanocyte precursors of the hair.

A subject of the invention is also a cosmetic treatment process for reducing and/or retarding the whitening of keratin fibers, in particular of head hair and/or bodily hair, and/or for promoting their natural repigmentation, characterized in that at least one plant of the species Punica granatum, or an extract of Punica granatum or a composition containing it, is administered orally to an individual.

The process according to the invention may thus be intended for reducing and/or retarding and/or preventing the whitening of head hair and/or bodily hair, and/or for promoting their natural repigmentation. The process thus enables the maintenance of a natural coloration and pigmentation of human hair, especially in the case of individuals over the age of 35, or even over the age of 45.

The oral intake of the extract of Punica granatum may be combined with a topical application to the skin or the integuments of an extract of Punica granatum that is identical to or different than that ingested.

According to one variant of the process according to the invention, at least one additional agent that is beneficial to head hair and/or bodily hair is also applied, in particular an agent for promoting the natural pigmentation or repigmentation of head hair and/or bodily hair and/or for reducing its whitening, or an agent for promoting hair growth or reducing hair loss, an agent for improving the quality of the hair fiber, such as an agent for reducing the breaking of the hair or for reinforcing the hair diameter, or alternatively an antidandruff agent or an agent for combating desquamative conditions of the hair. This application may be performed via any route, and especially topically or orally.

According to one particular embodiment of the invention, the compositions containing the extract of Punica granatum also contain at least one other agent for promoting the pigmentation of head hair or bodily hair, and/or at least one agent for promoting hair regrowth or reducing hair loss.

Among the additional agents that are suitable for implementing the invention, examples that may be mentioned include
agents for modifying the differentiation and/or proliferation and/or pigmentation of skin cells, such as retinol and esters thereof, vitamin D and derivatives thereof, cAMP modulators such as POMC derivatives, adenosine, forskolin and derivatives thereof, and prostaglandins and derivatives thereof;
extracts of microorganisms;
free-radical scavengers such as α-tocopherol or esters thereof, superoxide dismutases or mimetics thereof, certain metal-chelating agents or ascorbic acid and esters thereof;
antiseborrhea agents such as certain sulfur-containing amino acids, 13-cis-retinoic acid and cyproterone acetate;
other agents for combating desquamative conditions of the scalp, such as selenium disulfide, climbazole, undecylenic acid, ketoconazole, piroctone olamine (Octopirox) or ciclopiroctone (Ciclopirox);
plant extracts with propigmenting activity, for instance chrysanthemum extracts as described in FR 2 768 343 and the Sanguisorba extracts described in FR 2 782 920.

In particular, they may be active agents for stimulating hair regrowth and/or for promoting the slowing-down of hair loss. Mention may be made more particularly in a nonlimiting manner of:
nicotinic acid esters, especially including tocopheryl nicotinate, benzyl nicotinate and C₁-C₆ alkyl nicotinates, for instance methyl or hexyl nicotinate;
pyrimidine derivatives, for instance 2,4-diamino-6-piperidinopyrimidine 3-oxide or Minoxidil described in patents U.S. Pat. No. 4,139,619 and U.S. Pat. No. 4,596,812; Aminexil or 2,4-diaminopyrimidine 3-oxide described in WO 96/09048;
lipoxygenase inhibitors or cyclooxidase inducers for promoting hair regrowth, such as those described by the Applicant in European patent application EP 0 648 488;
calcium antagonists, for instance cinnarizine, nimodipine and nifedipine;
ATP-dependent potassium channel agonists such as cromakalim and nicorandil.

The additional agent may be chosen especially from the group comprising at least one extract of a plant chosen from Fragaria (strawberry), blackberry, raspberry, grape, walnut, Terminalia (in particular Belerica, Chebula and Arjuna), Caesalpinia spinosa, cypress, burnet, Sanguisorba officinalis or chrysanthemum (Chrysanthemum morifolium) or an agent chosen from tyrosine, L-dopa, pro-opiomelanocortin derivatives, adenosine, or forskolin or derivatives thereof.

According to one embodiment of the process according to the invention, at least one additional agent is applied topically to the skin and/or its appendages.

According to another embodiment of the process according to the invention, at least one additional agent, in particular at least one additional agent that is beneficial to the hair as defined hereinabove, especially for promoting natural repigmentation of the hair or for reducing its whitening, is administered orally.

Such additional active agents may be chosen especially from chlorine; grapeseed extracts; green tea extracts; trace elements such as zinc and selenium; vitamins such as vitamin A, vitamin E and vitamin C; catechins such as epicatechin, gallocatechin, epigallocatechin or epigallocatechin gallate; pine phytosterols.

Advantageously, the composition suitable for implementing the invention also contains at least one extract of a plant chosen from raspberry, blackberry, strawberry, grape, walnut, Terminalia (in particular Belerica, Chebula and Arjuna), and Caesalpinia spinosa.

The plant Punica granatum, extracts thereof or the composition containing them will be ingested regularly, daily, especially from 1 to 3 times per day. The taking of Punica granatum may be continued over time, for example for 2 to 4 weeks, but may be continued for 2 to 6 months without drawback.

The process according to the invention is a cosmetic process for improving the appearance of the hair, especially by retarding or reducing physiological whitening. The use of pomegranate extract according to the invention makes it possible to obtain good tolerance and good efficacy of the cosmetic treatment. Improved formulations are also obtained.

The invention also relates to an extract of at least one plant of the species Punica granatum for its use in preventing and/or treating canities.

The invention also relates to a combination product for simultaneous, separate or sequential use over time, characterized in that it comprises at least a component comprising a plant of the species Punica granatum and/or an extract of a plant of the species Punica granatum,
(ii) A second component comprising an additional active agent for promoting the natural pigmentation of the hair or for reducing its whitening, chosen from extracts of a plant chosen from Fragaria (strawberry), blackberry, raspberry, grape, walnut, Terminalia (in particular Belerica, Chebula and Arjuna) and Caesalpinia spinosa.

Other characteristics and advantages of the invention will emerge on reading the examples that follow.

### EXAMPLE I

### Measurement of the Activity of Human Hair Follicle Melanocytes

The activity of the melanocytes in human hair follicles is measured via the process described in the publication by Michelet et al. (Exp. Dermatol. 2008 Oct. 22).

Briefly, the test implementation steps were the following: human hair follicles are microdissected and cultured in vitro in William's E culture medium (Gibco-BRL) supplemented especially with glutamine, insulin, hydrocortisone and antibiotics.

The culture medium contains [¹⁴C] 2-thiouracil at a concentration of 3 μCi/ml.

For the “test” group of hair follicles (25 follicles), the culture medium is supplemented with the pomegranate extract. For the “control” group (25 follicles), the culture medium is supplemented with the vehicle (in the same amount as for the “test” group).

After 5 days, the hair follicles are washed with medium free of [¹⁴C] 2-thiouracil, and then lyzed by immersion in a solution of Soluene 350 raised to 50° C. A scintillant liquid is then added. The amount of [¹⁴C] 2-thiouracil present in the lyzates is measured using a Trilux 1450 Micro-Beta machine.

The pomegranate extract increases the incorporation of [¹⁴C] 2-thiouracil into the hair follicles.

### EXAMPLE II

### Correction of the Canities Biomarkers of Human Hair Follicles

In the hair follicle of a gray hair, certain mRNAs are overrepresented (overexpressed genes) and others are under-represented (underexpressed genes). These genes are canities biomarkers and their changes in expression are associated with the development of canities.

The level of expression of these canities biomarkers is analyzed by the standard technique of quantitative RT-PCR (Q-RT-PCR) in human hair follicles.

To perform the test, human hair follicles of gray hairs are microdissected and cultured in vitro in William's E culture medium (Gibco-BRL) supplemented especially with glutamine, insulin, hydrocortisone and antibiotics. For the “test” group of hair follicles (30 follicles), the culture medium is supplemented with the extract of Punica granatum. For the “control” group (30 follicles), the culture medium is supplemented with the vehicle (in the same amount as for the “test” group).

After the time necessary for revelation of the activity of the extract, in general from 24 hours to 5 days, the mRNAs of the hair follicles are extracted. The mRNAs of the same experimental condition are collated.

The level of expression of the mRNAs of interest is determined via a standard Q-RT-PCR method, for example using the QuantiTect SYBR® Green RT-PCR kit referenced 204243 from Qiagen, the measurement possibly being performed using the MylQ machine from Biorad.

The extract of Punica granatum corrects the changes in expression of certain canities biomarkers in the hair follicles of gray hair.

### EXAMPLES III

### Oral Compositions

### Example 1

### Formulation of Coated Tablet Type

| | mg/coated tablet |
|---|---|
| Active agent | |
| Extract of pomegranate | 300 |
| Excipient | |
| Microcrystalline cellulose | 70 |
| Encompress ™ | 60 |
| Magnesium stearate | 3 |
| Anhydrous colloidal silica | 1 |
| Coating agent | |
| Shellac | 5 |
| Talc | 60 |
| Sucrose | 50 |
| Polyvidone | 6 |
| Titanium dioxide | 0.3 |
| Colorant | 5 |

### Example 2

### Formulation of Gelatin Gel Capsule Type

| | mg/coated tablet |
|---|---|
| Active agent | |
| Extract of pomegranate | 300 |
| Excipient | |
| Starch | 200 |
| Magnesium stearate | 3 |

### Example 3

### Formulation of Single-Dose Gel Type

| | % |
|---|---|
| Active agent | |
| Extract of pomegranate | 8 |
| Excipient | |
| Sugar syrup | 50 |
| Maltodextrin | 15 |
| Xanthan gum | 1 |
| Sodium benzoate | 0.2 |
| Water | qs 100 |

### Example 4

### Formulation of Capsule Type

| | mg/capsule |
|---|---|
| Active agent | |
| Extract of pomegranate | 300 |
| Excipient | |
| Glycerol | 150 |
| Magnesium stearate | 3 |
| Water | qs 500 |

1. A method for reducing or preventing whitening of head hair, bodily hair, or both types of hair, the method comprising orally administering an extract of at least one plant of species Punica granatum to an individual.

2. The method of claim 1, wherein the extract is selected from the group consisting of a flower extract, a fruit extract, a seed extract and a peel extract of Punica granatum.

3. The method of claim 1, wherein the extract is selected from the group consisting of an aqueous extract, an alcoholic extract and an aqueous-alcoholic extract of Punica granatum.

4. The method of claim 1, comprising orally administering a composition comprising 0.01% to 99% by weight of the extract, based on a total weight of the composition.

5. A method of preventing canities, treating canities, or both preventing and treating canities, the method comprising orally administering an extract of at least one plant of species Punica granatum.

6. (canceled)

7. The method of claim 1, further comprising applying to an individual at least one additional agent selected from the group consisting of a head hair pigmentation promoting agent, a bodily hair pigmentation promoting agent, a hair loss reducing agent, a hair growth stimulating agent, a desquamative condition combating agent, a hair fiber quality improving agent, and a hair strand diameter reinforcing agent.

8. The method of claim 4, wherein the composition further comprises a hair pigmentation promoting agent or a hair regrowth promoting agent.

9. The method of claim 4, wherein the composition further comprises at least one extract of a plant selected from the group consisting of raspberry, blackberry, strawberry, grape, walnut, Terminalia, Belerica, Chebula, Arjuna and Caesalpinia spinosa.

10. The method of claim 1, further comprising topically applying to the hair or its appendages at least one additional agent.

11. The method of claim 1, wherein the extract is a total extract of Punica granatum.

12. The method of claim 4, wherein the composition comprises 0.01% to 90% by weight of the extract.

13. The method of claim 4, wherein the composition comprises 0.1% to 50% by weight of the extract.

14. The method of claim 4, wherein the composition comprises 5% to 50% by weight of the extract.

15. The method of claim 4, wherein the composition comprises 40% to 50% by weight of the extract.

16. The method of claim 1, comprising administering 10 mg to 1000 mg of the extract per day.

17. The method of claim 1, comprising administering 200 mg to 500 mg of the extract per day.

18. The method of claim 1, comprising administering the extract 1 to 3 times per day.

19. The method of claim 1, comprising administering the extract 1 to 3 times per day, for 2 to 4 weeks.

20. The method of claim 1, comprising administering the extract 1 to 3 times per day, for 2 to 6 months.

21. The method of claim 17, comprising administering the extract 1 to 3 times per day, for 2 to 6 months.
